(19) United States
(12) Patent Application Publication
(10) Pub. No.: US 2020/0046658 A1
(43) Pub. Date: Feb. 13, 2020

(54) PHARMACEUTICAL COMPOSITION FOR INDUCING EXERCISE MIMETIC EFFECT

(71) Applicant: CELLVERTICS CO., LTD., Seoul (KR)

(72) Inventors: Hong Seog SEO, Seoul (KR); Eung Ju KIM, Seoul (KR); Yong Jik LEE, Incheon (KR); Hyeon Soo KIM, Seoul (KR)

(73) Assignee: CELLVERTICS CO., LTD., Seoul (KR)

(21) Appl. No.: 16/591,713

(22) Filed: Oct. 3, 2019

Related U.S. Application Data
(62) Division of application No. 15/762,828, filed on Mar. 23, 2018.

(30) Foreign Application Priority Data
Sep. 25, 2015 (KR) 10-2015-0136009
Sep. 26, 2016 (KR) 10-2016-0122779
Sep. 26, 2016 (KR) 10-2016-0122780

(51) Int. Cl. A61K 31/165; A61P 3/10; G01N 33/573; A61P 3/04; G01N 33/94

(52) U.S. Cl. CPC A61K 31/165; A61P 3/10; G01N 33/573; G01N 2333/70571; G01N 33/9406; G01N 2500/10; G01N 2800/04; A61P 3/04

(57) ABSTRACT

The present disclosure relates to a pharmaceutical composition for inducing an exercise mimetic effect, which contains an α₁-adrenergic receptor agonist as an active ingredient, and a method for screening a drug for inducing an exercise mimetic effect using the α₁-adrenergic receptor agonist.

The α₁-adrenergic receptor agonist of the present disclosure increases the expression of p-AMPK, PPARδ and PGC-1α, which play key roles in maintaining and regulating energy metabolic activity in vivo, thereby increasing glucose uptake into skeletal muscle cells, suppressing adipocyte differentiation and lipid accumulation, reducing abdominal fat and body weight as well as regulating mitochondrial metabolic disorders and suppressing inflammatory responses. Accordingly, the α₁-adrenergic receptor agonist can be usefully used to prevent and treat diseases requiring AMPK activation (metabolic diseases, cardiovascular diseases, inflammatory disease, etc.).

### CROSS-REFERENCE TO RELATED APPLICATION

This application is a divisional of U.S. patent application Ser. No. 15/762,828 filed Mar. 28, 2018 which is a is a U.S. National Stage Application of International Application No. PCT/KR2016/010781, filed on Sep. 26, 2016 which claims priority to and the benefit of Korean Patent Applications Nos. 10-2015-0136009, 10-2016-0122779 and 10-2016-0122780 filed on Sep. 25, 2015, Sep. 26, 2016, and Sep. 26, 2016, respectively, the disclosures of which are incorporated herein by reference in their entireties.

### BACKGROUND

### 1. Field of the Invention

The present disclosure relates to a pharmaceutical composition for inducing an exercise mimetic effect, which contains an α₁-adrenergic receptor agonist as an active ingredient, and a method for screening a drug for inducing an exercise mimetic effect using the α₁-adrenergic receptor agonist.

### 2. Description of Related Art

Exercise training improves exercise tolerance by activating the remodeling program causing change in the phenotype of skeletal muscle and moderate exercise is effective in improving the pathological conditions of metabolic diseases, heart diseases, etc.

Especially, AMPK (AMP-activated protein kinase), PPAR-δ (peroxisome proliferator-activated receptor-δ) and PGC-1α (peroxisome proliferator-activated receptor gamma coactivator-1α) are known as important factors involved in the change in the phenotype of skeletal muscle for exercise tolerance.

AMPK, which is a heterotrimeric complex consisting of α, β and γ subunits, is activated by phosphorylation during muscle contraction and exercise by LKB1 and CaMKK (Ca^{2α}/calmodulin-dependent kinase kinase) and acts as a major regulator in the cellular/organ metabolisms for glucose homeostasis, appetite and exercise physiology. PPAR-δ plays a critical role in the transcriptional regulation of skeletal muscle metabolism. PGC-1α is involved in energy metabolism as a regulator of mitochondrial biosynthesis and function. It is a transcriptional coactivator that regulates the genes activated by the exercise tolerance of skeletal muscle.

AMPK is known to be capable of targeting several transcriptional programs at the same time which are regulated by substrates such as PPAR-δ and PGC-1α and induce genetic effects similar to that of exercise. In this regard, it is reported that drugs targeting the AMPK/PPARδ signaling pathway can become a new pharmaceutical strategy for reprogramming muscles (Cell 2008, vol. 134, pp. 405-415).

In addition, it is reported that some drugs facilitating AMPK activation such as A-769662, metformin, 5-aminoimidazole-4-carboxamide-1-β-D-ribofuranoside (AICAR) and resveratrol exert therapeutic effects for heart failure. The effect of protecting the heart by AMPK can be achieved through important functions such as decreased production of reactive oxygen species in the cytoplasm, inhibition of angiotensin II activity, phosphorylation of cardiac troponin I, activation of PGC-1α, regulation of eNOS-NAD(P)H oxidase expression, regulation of estrogen-related receptors, regulation of energy balancing and intracardiac signaling, etc. The activation of AMPK can improve the cardiac muscle function directly or indirectly.

As described, because AMPK activation (phosphorylation) and the factors related thereto (PPAR-δ and PGC-1α) induce exercise mimetic effects such as glucose uptake in skeletal muscle, maintenance of homeostasis in energy metabolism, enhancement of cardiac function, etc., there have been consistent demands on drugs that activate AMPK as a target for metabolic diseases, cardiovascular diseases, inflammatory diseases, etc. that can be treated by the exercise mimetic effect.

### SUMMARY OF THE INVENTION

The inventors of the present disclosure have found out that hypertension, metabolic diseases such as obesity and diabetes, etc., heart diseases and inflammatory diseases can be improved by activating the α₁-adrenergic receptor and have researched its mechanism of action. As a result, they have found out that the activation of the α₁-adrenergic receptor increases the expression of activated AMPK, PPAR-δ and PGC-1α and, through this, can induce an exercise mimetic effect in multiple organs such as skeletal muscle, cardiac muscle, liver, etc. and have completed the present disclosure.

The present disclosure is directed to providing a pharmaceutical composition for inducing an exercise mimetic effect, which contains an α₁-adrenergic receptor agonist or a pharmaceutically acceptable salt thereof as an active ingredient, and a therapeutic method using the same.

The present disclosure is also directed to providing a method for screening a drug for inducing an exercise mimetic effect using the α₁-adrenergic receptor agonist.

However, the technical problems to be solved by the present disclosure is not limited to those described above. Other technical problems not mentioned will be more clearly understood by those skilled in the art from the following description.

The present disclosure relates to a pharmaceutical composition for inducing an exercise memetic effect, which contains an α₁-adrenergic receptor agonist or a pharmaceutically acceptable salt thereof as an active ingredient.

The present disclosure also relates to a method for inducing an exercise mimetic effect, which includes a step of administering an α₁-adrenergic receptor agonist or a pharmaceutically acceptable salt thereof to a subject.

The present disclosure also relates to a use of an α₁-adrenergic receptor agonist or a pharmaceutically acceptable salt thereof for inducing an exercise mimetic effect.

In a specific exemplary embodiment of the present disclosure, the α₁-adrenergic receptor agonist induces the activation of AMPK.

In another specific exemplary embodiment of the present disclosure, the α₁-adrenergic receptor agonist induces the expression of PPAR-δ or PGC-1α.

In another specific exemplary embodiment of the present disclosure, the α₁-adrenergic receptor agonist is midodrine.

In another specific exemplary embodiment of the present disclosure, the exercise mimetic effect is an effect of treating a disease requiring the activation of AMPK.

The present disclosure also relates to a method for screening a drug for inducing an exercise mimetic effect using an α₁-adrenergic receptor agonist.

In an exemplary embodiment of the present disclosure, the screening method includes: (a) a step of treating a cell with an α₁-adrenergic receptor agonist in vitro; and (b) a step of measuring the expression of phosphorylated AMPK (AMP-activated protein kinase), PPAR-δ (peroxisome proliferator-activated receptor-δ) or PGC-1α (peroxisome proliferator-activated receptor gamma coactivator-1α).

In another exemplary embodiment of the present disclosure, the screening method further comprises (c) a step of selecting the agonist as a drug when the expression of phosphorylated AMPK, PPAR-δ or PGC-1α is increased as compared to a group not treated with the agonist.

In another exemplary embodiment of the present disclosure, in the step (b), the expression is measured by western blotting, antigen immunoprecipitation, ELISA, mass spectrometry, RT-PCR, competitive RT-PCR, real-time RT-PCR, RPA (RNase protection assay) or northern blotting.

In another exemplary embodiment of the present disclosure, in the step (a), the cell is the cell of a tissue selected from a group including skeletal muscle, cardiac muscle, liver, fat and pancreas.

In another exemplary embodiment of the present disclosure, the exercise mimetic effect is an effect of treating a disease selected from a group including a metabolic disease, a cardiovascular disease and an inflammatory disease.

In another exemplary embodiment of the present disclosure, the metabolic disease is a disease selected from a group including hypertension, hyperlipidemia, diabetes, obesity, arteriosclerosis and fatty liver.

An α₁-adrenergic receptor agonist of the present disclosure increases the expression of p-AMPK, PPARδ and PGC-1α, which play key roles in maintaining and regulating energy metabolic activity in vivo, thereby increasing glucose uptake into skeletal muscle cells, suppressing adipocyte differentiation and lipid accumulation, reducing abdominal fat and body weight as well as regulating mitochondrial metabolic disturbances and suppressing inflammatory responses. Accordingly, the α₁-adrenergic receptor agonist can be usefully used to prevent and treat diseases requiring AMPK activation (metabolic diseases, cardiovascular diseases, inflammatory disease, etc.).

A composition of the present disclosure can be used directly for clinical application because a drug free from side effects and proven safety is used (drug repositioning).

And, according to a method of the present disclosure, a drug inducing an exercise mimetic effect can be screened conveniently with high precision in vitro.

### BRIEF DESCRIPTION OF THE DRAWINGS

FIGS. 1A and 1B show a western blot result showing that the expression of activated (phosphorylated) AMPK (p-AMPK) and PPARδ proteins is increased in skeletal muscle, cardiac muscle and liver cells when the α₁-adrenergic receptor (α₁-AR) is activated by midodrine.

FIG. 2A shows a result of investigating the expression of α₁-AR protein in the skeletal muscles of a 4-week-old basal control group rat (I), a midodrine-administered rat (II), an atenolol-administered rat (III) and an 8-week-old unadministered control group rat (IV) by western blotting and FIGS. 2B-2D show a result of investigating the expression of AMPK, PPARδ and PGC1α proteins in the cardiac muscles, skeletal muscles and livers, respectively, of the rat groups by western blotting.

FIGS. 3A and 3B show a northern blot result of investigating the mRNA expression level of genes associated with vasoconstriction, nitric oxide production, oxidative stress and inflammation, respectively, in the aortic tissues (FIG. 3A) and the cardiac muscle tissues (FIG. 3B) of a 4-week-old basal rat (I), a midodrine-administered rat (II), an atenolol-administered rat (III) and an 8-week-old unadministered control group rat (IV).

FIG. 4A shows a result of measuring the enzyme activity of SDH (succinate dehydrogenase) in the skeletal muscles of a 4-week-old basal rat (I), a midodrine-administered rat (II), an atenolol-administered rat (III) and an 8-week-old unadministered control group rat (IV) and FIG. 4B shows an immunohistochemical staining result of cytochrome c oxidase for the skeletal muscle tissues of the above groups.

FIG. 5 shows a result of measuring the ATP level in the cardiac muscles, skeletal muscles and livers of a 4-week-old basal rat (I), a midodrine-administered rat (II), an atenolol-administered rat (III) and an 8-week-old unadministered control group rat (IV) by ELISA.

FIG. 6 shows a result of investigating the effect of midodrine on glucose uptake by insulin in mouse skeletal muscle cells (C2C12 cells).

FIG. 7A shows that fat synthesis and accumulation are decreased in differentiated adipocytes after administration of midodrine and the effect is suppressed after administration of a PPARδ antagonist and FIG. 7B shows a result of investigating the mechanism of the effect of midodrine on the suppression of fat synthesis and accumulation in the adipocytes through expression of PPARδ, p-AMPK and PGC-1α proteins by western blotting.

FIGS. 8A and 8B show a result of investigating the effect of midodrine on body weight and abdominal fat weight.

FIGS. 9A and 9B show a result of investigating the effect of midodrine on the expression of the mannose receptor (MR) and hexokinase II in inflammation-associated macrophages. FIG. 9C shows an immunohistochemical staining result for investigating the mannose receptor expression of macrophages localized in the subcapsular portion of the spleens in a 4-week-old basal rat (I), a midodrine-administered rat (II), an atenolol-administered rat (III) and an 8-week-old unadministered control group rat (IV). It can be seen that the expression of the mannose receptor is significantly increased in the midodrine-administered group (II) as compared to other animal groups.

FIGS. 10A and 10B show a western blot result showing that the expression of p-AMPK (A) and PPAR-δ (B) proteins in mouse skeletal muscle cells (C2C12) when the α₁-adrenergic receptor (α₁-AR) is activated by Compound 5.

FIGS. 11A and 11B show a western blot result showing that the expression of p-AMPK (A) and PPAR-δ (B) proteins in mouse skeletal muscle cells (C2C12) when the α₁-adrenergic receptor (α₁-AR) is activated by Compound 7.

FIGS. 12A-12C show a western blot result showing that the expression of p-AMPK (A), PPAR-δ (B) and PGC-1α (C) proteins in mouse skeletal muscle cells (C2C12) when the α₁-adrenergic receptor (α₁-AR) is activated by Compound 8.

FIGS. 13A-13C show a western blot result showing that the expression of p-AMPK (A), PPAR-δ (B) and PGC-1α (C) proteins in mouse skeletal muscle cells (C2C12) when the α₁-adrenergic receptor (α₁-AR) is activated by Compound 9.

FIGS. 14A-14C show a western blot result showing that the expression of p-AMPK (A), PPAR-δ (B) and PGC-1α (C) proteins in mouse skeletal muscle cells (C2C12) when the α₁-adrenergic receptor (α₁-AR) is activated by Compound 10.

### DETAILED DESCRIPTION OF THE INVENTION

The present disclosure provides a pharmaceutical composition for inducing an exercise mimetic effect, which contains an α₁-adrenergic receptor agonist or a pharmaceutically acceptable salt thereof as an active ingredient.

The present disclosure has been devised based on the finding that the α₁-AR agonist can exert an exercise mimetic effect in multiple organs such as skeletal muscle, cardiac muscle, liver, etc. through AMPK activation in addition to its intrinsic effect of improved cardiac muscle contraction through stimulation of α₁-AR.

In the present disclosure, the α₁-adrenergic receptor agonist is not specially limited as long as it is a substance acting on and activating the α₁-adrenergic receptor. There are three subtypes α₁, α₂ and β of the adrenergic receptor. In the present disclosure, any known compound that activates the α₁-type receptor can be used without limitation. Examples may include midodrine, analogues thereof or pharmaceutically acceptable salts thereof.

The midodrine compound is marketed under the brand names Amatine, ProAmatine, Gutron, etc. Its IUPAC name is (R/S)—N-[2-(2,5-dimethoxyphenyl)-2-hydroxyethyl]glycinamide and is represented by Chemical Formula I.

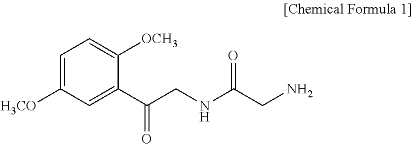 [SMILES: COc1ccc(OC)c(C(=O)CNC(=O)CN)c1]

Midodrine is a prodrug which is changed into a target compound in vivo after being administered. After the administration, it is changed into the active metabolite, desglymidodrine, which activates the α₁-adrenergic receptor, thereby inducing AMPK activation and PPAR-δ or PGC-1α expression and finally inducing an exercise mimetic effect.

The compound represented by Chemical Formula 1 may form “a pharmaceutically acceptable salt”. A suitable pharmaceutically acceptable salt is one commonly used in the art to which the present disclosure belongs, such as an acid addition salt, and is not specially limited. Specifically, the acid that may be used in the pharmaceutically acceptable acid addition salt may be, for example, an inorganic acid such as hydrochloric acid, sulfuric acid, nitric acid, phosphoric acid, perchloric acid or bromic acid or an organic acid such as acetic acid, methanesulfonic acid, ethanesulfonic acid, p-toluenesulfonic acid, fumaric acid, maleic acid, malonic acid, phthalic acid, succinic acid, lactic acid, citric acid, gluconic acid, tartaric acid, salicylic acid, malic acid, oxalic acid, benzoic acid, embonic acid, aspartic acid or glutamic acid. An organic base that may be used to prepare an organic base addition salt may be, for example, tris(hydroxymethyl)methylamine, dicyclohexylamine, etc. An amino acid that may be used to prepare an amino acid addition base may be a naturally occurring amino acid such as alanine, glycine, etc.

Also, in the present disclosure, the midodrine “analogue” is not specially limited as long as it is a compound having a structure similar to that of midodrine and exhibiting an effect comparable to that of midodrine. Examples may include the following Compounds 5, 7, 8, 9 and 10.

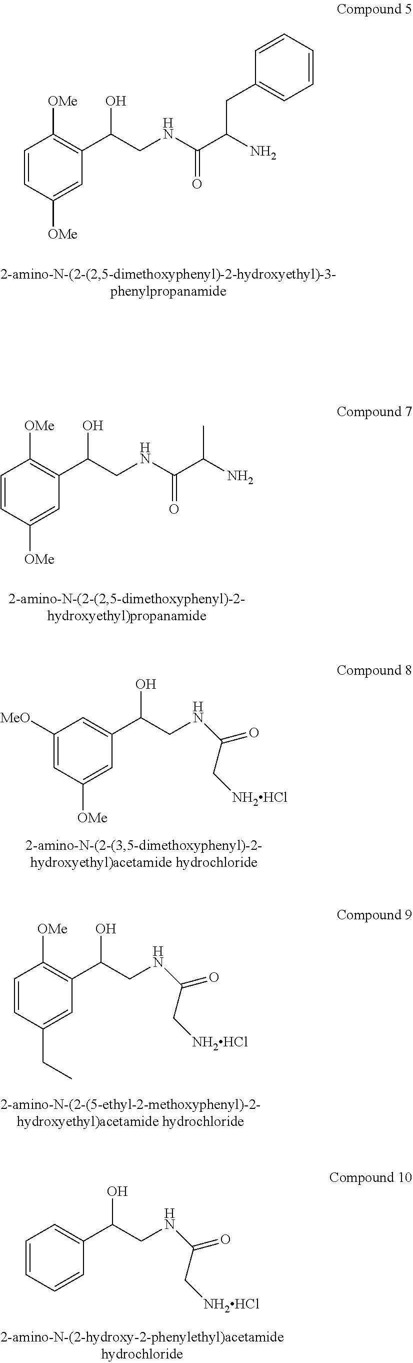

In the present disclosure, the α₁-adrenergic receptor agonist may achieve an exercise mimetic effect by inducing AMPK activation and PPAR-δ or PGC-1α expression.

AMPK acts as an energy sensor which senses energy state in vivo and maintains it at a constant level. When the energy in a cell is decreased due to, for example, metabolic stress or exercise, i.e., when the AMP/ATP ratio is increased due to depletion of ATP, AMPK is activated and facilitates the processes consuming ATP (e.g., fatty acid oxidation or glycolysis). AMPK activation induces metabolically important results in major target organs (liver, muscles, fats or pancreas). It is known to inhibit fatty acid and cholesterol synthesis and facilitate fatty acid oxidation in the liver, facilitate fatty acid oxidation and glucose uptake in skeletal muscles, inhibit fat synthesis in adipocytes and facilitate insulin secretion in pancreatic β cells.

PPAR-δ is known to facilitate catabolic energy metabolism in cells by regulating AMPK and play an essential role in maintaining metabolic balance (homeostasis) such as anti-inflammation, inhibition of insulin resistance, etc.

PGC-1α is a major regulator of mitochondrial biogenesis. It is known that its expression is induced by severe metabolic changes such as exercise, starvation, cold, etc. and is regulated by AMPK, PPAR-δ, NAD-dependent deacetylase sirtuin-1 (SIRT1), etc.

In the present disclosure, the exercise mimetic effect means a physiological effect exerted during exercise such as improvement in cardiac function (increase in contractile force), increase in the insulin sensitivity and oxidative phosphorylation of muscles, decrease in cholesterol, decrease in fat accumulation and body weight, decrease in blood inflammation, etc., although not being specially limited thereto.

In the present disclosure, the disease requiring AMPK activation refers to various diseases that can be cause by deactivation of AMPK without special limitation. For example, it may be a metabolic disease, a cardiovascular disease, an inflammatory disease, etc.

In the present disclosure, the “metabolic disease” refers to a disease caused by abnormal metabolism of glucose, fats, proteins, etc. Examples may include hyperlipidemia, diabetes, obesity, arteriosclerosis, fatty liver, etc.

In the present disclosure, the “pharmaceutical composition” may further include an existing therapeutically active ingredient, an adjuvant, a pharmaceutically acceptable carrier, etc. The pharmaceutically acceptable carrier includes a saline, sterilized water, a Ringer's solution, a buffered saline, a dextrose solution, a maltodextrin solution, glycerol, ethanol, etc.

The composition may be formulated as an oral formulation such as a powder, a granule, a tablet, a capsule, a suspension, an emulsion, a syrup, an aerosol, etc., a formulation for external application, a suppository or a sterilized injectable solution according to commonly employed methods.

In the present disclosure, it is obvious to those skilled in the art than an “administration dosage” can be controlled variously depending on the body weight, age, sex, health condition and diet of a patient, number of administrations, administration method, excretion rate, the severity of a disease, etc.

In the present disclosure, a “subject” means a subject in need of treatment of a disease, more specifically a mammal such as human, non-human primates, mouse, rat, dog, cat, horse, cow, etc.

In the present disclosure, a “pharmaceutically effective amount” is determined by the kind and severity of a disease, the age and sex of a patient, administration time, administration route, excretion rate, treatment period, the drug(s) used in combination and other factors well known in the medical field. It can be easily determined by those skilled in the art as an amount that can achieve the maximum effect without a side effect in consideration of the above-described factors.

The composition of the present disclosure is not limited in “administration method” as long as it can reach the target tissue. For example, it may be administered orally, intraarterially, intravenously, transdermally, intranasally, transbronchially or intramuscularly. A daily administration dosage is about 0.0001-100 mg/kg, specifically 0.001-10 mg/kg, and the administration may be made once or several times a day.

In the present disclosure, it has been found out that the α₁-AR agonist improves the cardiac function by increasing PPAR-δ and PGC-1α expression independently from activating AMPK by stimulating α₁-AR. It is because it increases the rate of heart contraction and relaxation like exercise training.

Accordingly, it is thought that the α₁-AR agonist directly stimulates heart contraction and indirectly improves cardiac function through exercise mimetic AMPK and PPARδ activation by stimulation of α₁-AR in multiple organs including the heart, muscles and liver, thereby contributing to improvement in exercise tolerance in vivo.

The present invention discloses that stimulation of α₁-AR leads to the exercise mimetic effect on the heart through the AMPK and PPARδ activation. Also, the present invention discloses that the exercise mimetic effect activated through increased PPARδ and PGC-1α expression with by AMPK is working to the heart independently.

Specifically, in the present disclosure, the effect of α₁-AR stimulation on the expression of the genes involved in exercise mimetic effect on cardiac muscle cells and skeletal muscle cells was investigated, and the same effect on the cardiac and skeletal muscles and the liver in vivo was compared using a rat (SHR) which is an animal model for metabolic syndrome in human.

Also, the effect of α₁-AR stimulation on the cardiac function/size of the heart, inflammatory cytokines, level of reactive oxygen species, adiponectin, ATP level, SDH (succinate dehydrogenase) activity, fat amount, angiotensin II AT-1 receptor expression, etc. was investigated.

As a result, it was elucidated that, even in the early age when the SHR rat shows consistent increase in blood pressure, stimulation of α₁-AR by the α₁-AR agonist induces activation and increased expression of AMPK, PPAR-δ and PGC-1α in the heart and skeletal muscles and increases heart cardiac contractility without cardiac hypertrophy or additional blood pressure increase.

In addition, it was confirmed that the α₁-AR stimulation results in decreased level of inflammatory cytokines, ROS and cholesterol in blood, and that the ratio of-PPARδ and PGC-1α activation to AMPK stimulation is higher in cardiac muscles than in skeletal muscles and liver. These results are associated exercise mimetic effect through AMPK-PPARδ activation, independent from the muscle contraction effect. Also, it is the first case showing that the enhanced cardiac muscle contraction by pharmacological α₁-AR stimulation is contributed by the AMPK-PPARδ-PGC1α activation.

Also, in the present disclosure, it was elucidated that the α₁-AR agonist is more effective than other drugs inducing the exercise mimetic effect (e.g., atenolol). It is because the α₁-AR agonist exhibits the dual effect of improving cardiac contractility whereas other drugs exhibit only the exercise mimetic effect without direct cardiac contraction. In addition, whereas the other drugs inducing the exercise mimetic effect are used in combination to enhance their respective functions, the α₁-AR agonist can exert the exercise mimetic effect in multiple organs at the same time.

Also, in the present disclosure, it was confirmed that, although the rats treated with midodrine showed increase in left ventricular ejection fraction as that of the animals treated with atenolol, there was a difference in the level of phosphorylated AMPK in the heart between the experimental groups. Although α-1 AR provides a favorable effect for the heart, the degree of α-1 AR stimulation is important in terms of long-term prognosis because the extremely enhanced cardiac α-1 AR drive can induce pathological remodeling in contractility disturbance, gradual fibrosis and reactivation of protein genes of fibroblasts.

Also, in the present disclosure, it was confirmed that cardiac hypertrophy did not occur despite the α₁-AR stimulation. It may be due to the effect of AMPK on inhibition of angiotensin II, stimulating autophagy, etc. In addition, the left ventricular mass was smaller for the midodrine-treated group than the atenolol-treated group, which can be explained with the difference in cardiac AT1 expression.

Also, in the present disclosure, it was confirmed through in-vitro and in-vivo experiments for the first time that, regardless of muscle exercise, α₁-AR stimulation activates AMPK, PPAR-δ and PGC1α in skeletal muscles. It has been already reported that the expression of these exercise-related genes is one of the adaptive responses for maintaining endurance exercise.

In addition, in the present disclosure, it was confirmed that the α₁-AR stimulation leads to decreased ROS level, increased activation of AMPK, PPAR-δ and PGC1α in the liver, and induced a systemic effect for metabolic/biochemical/inflammatory responses and significant decrease in total cholesterol, LDL-cholesterol and HDL-cholesterol.

Also, in the present disclosure, it was confirmed that the α₁-AR stimulation leads to increased glucose uptake by insulin, inhibited fat differentiation into adipocytes and fat synthesis/accumulation, body weight, abdominal fat amount, and inflammation. Therefore, it can be seen that the α₁-AR stimulation is effective in such diseases as diabetes, obesity, inflammation, etc.

Also, in the present disclosure, it was confirmed that, in addition to the α₁-adrenergic receptor agonist midodrine, various newly synthesized compounds similar thereto can induce AMPK activation and PPAR-δ and PGC-1α expression in skeletal muscle cells by stimulating α₁-AR, thereby inducing an exercise mimetic effect.

Hereinafter, the present disclosure will be described in detail through examples. However, the following examples are for illustrative purposes only and it will be apparent to those of ordinary skill in the art that the scope of the present disclosure is not limited by the examples.

### EXAMPLES

### Example 1: Methods

1-1. Materials

The TRIzol reagent was purchased from Invitrogen (CA, USA). The Power cDNA synthesis kit and the Maxime PCR PreMix kit were purchased from iNtRON Biotechnology (Seongnam-si, Gyeonggi-do, Korea). The PREP™ protein extraction solution and a prestained protein size marker were purchased from iNtRON Biotechnology. The anti-AMPKα (α subunit) and anti-phospho-AMPKα (phosphorylated at Thr172) primary antibodies were purchased from Cell Signaling Technology, Inc. (Danvers, Mass., USA). The anti-rabbit secondary antibody was purchased from Santa Cruz Biotechnology (Santa Cruz, Calif., USA). The Clarity Western ECL Substrate kit was purchased from Bio-Rad (Hercules, Calif., USA). An X-ray film was purchased from Agfa (Mortsel, Belgium) and the development/fixation kit was purchased from Kodak (Rochester, N.Y., USA). The rat adiponectin detection ELISA kit was purchased from Abcam (Cambridge, UK).

1-2. Effect of α₁-AR Stimulation on AMPK in Skeletal Muscle/Cardiac Muscle Cells In Vitro

In order to investigate the effect of α₁-AR stimulation in on the exercise mimetic effect of muscles in vitro, the expression of AMPK, which is the representative protein increased during exercise, was investigated first. Then, in-vivo animal experiment was conducted as follows on the assumption that the expression of PPARδ and PGC-1α will also be increased in the skeletal muscle, cardiac muscle and liver.

1-3. Cell Culturing and Animal Experiment

Cell Culturing

C2C12 cells (mouse skeletal cell line) were seeded onto a 6-well plate in a CO₂ incubator at 37° C. and cultured in a DMEM medium containing 10% FBS and 1% antibiotics to about 80% confluency. After replacing the medium with one containing 1% FBS, differentiation was conducted for 3 days. Then, after replacing the medium with one containing 1% FBS and then treating with a drug, experiment was conducted 24 hours later.

Animal Experiment

Spontaneously hypertensive rat (SHR) is an animal model in which hereditary hypertension is expressed. It is known that the hypertension which is the most similar to human primary hypertension is expressed. In SHR, blood pressure begins to increase around 4-6 weeks of age and hypertension is developed seriously at 8-12 weeks. SHR is widely used in studies as an animal mode of primary hypertension, especially hypertension with cardiac lesions, because it is frequently accompanied damage to the hypertensive target organs, such as cardiac hypertrophy, heart failure, renal disorder, etc.

3-week-old spontaneously hypertensive rats (SHRs) were kept under a standardized condition (21° C., 41-62% humidity) with periodic light/dark (10/14 hour) cycles and were freely allowed to access water and laboratory feed. All the animal experiments were approved by the Korea University Institutional Animal Care and Use Committee (KUIACUC-2012-100) and were in accordance with the Guideline for the Care and Use of Laboratory Animals.

After 1 week of acclimation, the rats were divided into four groups (6 rats per group) as follows: group I (basal control group, sacrificed at 4 weeks of age), group II (administered with midodrine for 4 weeks), group III (administered with atenolol for 4 weeks), group IV (control group with no drug administration for 4 weeks). The group I and group IV were given standard feed containing no drug (K—H4 pellet, Ssniff), the group II was given the same feed together with drinking water containing midodrine (0.2 mg/kg/day) and the group III was given the same feed together with drinking water containing atenolol (1 mg/kg/day).

Blood pressure was measured with 7-day intervals by tail-cuff plethysmography using the Visitech BP2000 system (Visitech Systems Inc.).

The animals of the group I were anesthetized at 4 weeks of age while the rats of other groups were anesthetized at 8 weeks of age after being treated for 4 weeks. Blood samples were taken from the inferior vena cava and the heart, aorta, liver, skeletal muscle and visceral fat were excised cleanly and then weighed. The recovered organs were kept in a refrigerator at −80° C. or in 10% formalin for fixation.

1-4. Echocardiographic Examination

After anesthetization by intramuscular injection of Zoletil (8 mg/kg) and xylazine (2 mg/kg), the rats were laid on their left sides and M-mode echo images were obtained. The examination was conducted using Vivid 7 (GE Medical Systems, Milwaukee, Wis., USA) equipped with a 12-MHz transducer. After acquiring optimized 2-dimensional short-axis images for the left ventricle at the papillary muscle level, M-mode tracing and echocardiographic images were recorded at a speed of 100 mm/s. The wall thickness, volume and mass of the heart was measured from at least three consecutive cardiac cycles on the M-mode tracing using as suggested by the American Society for Echocardiography.

1-5. Measurement of Blood Biochemistry, Inflammatory Markers, Reactive Oxygen Species, ATP and Adiponectin

The concentration of total cholesterol, HDL cholesterol, LDL cholesterol and triglyceride was measured by enzymatic colorimetry (Roche Diagnostics GmbH; Mannheim, Germany). Also, serum assays for inflammatory markers were conducted using a 4-plex cytokine Milliplex panel (Millipore Corporation, Billerica, Mass., USA) as recommended for cytokines (interleukin (IL)-1α, IL-1β, IL-6, IL-4, IL-10 and TNF-α). Acquisition was performed on a Luminex 100 platform and data analysis was carried out using the Multiplex analyzer. Reactive oxygen species and adiponectin were measured using the ELISA kit (MBS815494, Mybiosource). The measured blood adiponectin level was calibrated for the visceral fat weight (g).

1-6. ELISA for Measurement of ATP and ROS in Tissue

0.02 g of liver tissue was homogenized in 500 μL of PBS. The homogenate was centrifuged at 1500×g (or 5000 rpm) for 15 minutes and the supernatant was subjected to measurement. After pouring 100 μL of a reference material or sample to an adequate well of an antibody-precoated microtiter plate, 10 μL of the residual solution was added to the sample. After adding 50 μL of a conjugate to each well and mixing, the plate was covered with a lid and incubated at 37° C. for 1 hour. After adding 50 μL of substrate A or B to each well, incubation was conducted at 37° C. for 15 minutes. After adding a stop solution to each well, optical density was measured at 450 nm using a microplate reader.

1-7. Measurement of SDH (Succinate Dehydrogenase) Activity in Skeletal Muscle

After adding 10 μL of a protease inhibitor cocktail to 1 mL of PBS per well and making the volume 500 μL, 410 μL of a culture solution prepared by mixing 1 M PBS (25 μL×25=625 μL), 0.2 M sodium succinate (125 μL×25=3125 μL), NBT (25 μL×25=625 μL) and D.W. (235 μL×25=5875 μL) was heated to 37° C. 20 minutes before conducting reaction. After adding 500 μL of PBS to 0.02 g of skeletal muscle tissue, the tissue was crushed in sort time to prevent enzymes from being broken down. After centrifugation at 13000 rpm and 4° C. for 5 minutes, the supernatant was transferred to a fresh tube. 410 μL of the culture solution was transferred to a tube in advance and then enzymatic reaction was conducted by adding 90 μL of the sample. After adding 410 μL of D.W. to another tube and then adding 90 μL of the sample, the absorbance of the diluted solution was measured. The enzymatic reaction tube and another tube were put in a water bath at 37° C., respectively, and then subjected to reaction for 30 minutes. After terminating the reaction by putting the tubes in ice, 200 μL of each solution was transferred to a 96-well plate and absorbance was measured at 550 nm. The enzyme activity of succinate dehydrogenase (SDH) was calculated from the result according to the following equation.

Enzyme activity=(absorbance of enzymatic reaction solution−absorbance of diluted enzyme solution)/protein quantity (Bradford 595 nm)

1-8. Reverse Transcription Polymerase Chain Reaction (RT-PCR) for Quantification of mRNA in Aorta and Heart

Full-length RNA was extracted using the TriZol reagent according to the manufacturer's instructions. Complementary DNA was synthesized from the full-length RNA using the Power cDNA Synthesis kit and polymerase chain reaction was conducted for angiotensin II type I receptor (AT1R), endothelial nitric oxide synthase (eNOS), superoxide dismutase (SOD), gp91-phox (NADPH), tumor necrosis factor-alpha (TNF-α) and GAPDH using the PCR Premix kit.

1-9. Western Blot

The protein content of the extract was measured by the Bradford's method. The extracted proteins (20-30 μg) were loaded on a 10% SDS-PAGE gel. Western blot assay was conducted using primary antibodies for AMPKα, phosphorylated AMPKα, PPARδ and PGC-1α. Images were obtained manually using the Kodak GBX developer and a fixative reagent.

1-10. Statistical Analysis

All the recorded blood pressure measurements were analyzed [4- to 8-week-old three animal groups]. The blood pressure was compared by ANOVA and was regarded significant when p<0.05. Continuous variables were represented by mean±standard deviation (SD). The overall difference between four groups was analyzed by the Kruskal-Wallis test. The difference between two groups was evaluated by the Mann-Whitney U-test. p-values smaller than 0.05 were regarded statistically significant. All statistical analysis was conducted using SPSS (ver. 20.0; SPSS Inc., Chicago, Ill., USA).

### Example 2: Results

2-1. Effect of α₁-AR Stimulation on AMPK Phosphorylation in Skeletal Muscle/Cardiac Muscle Cells In Vitro

In order to investigate the effect of α₁-AR (α₁-adrenergic receptor) stimulation on AMPK and phosphorylated (activated) AMPK in vitro, rat skeletal muscle cells (L6) and mouse cardiac muscle cells (HL1) were treated with the α₁-AR agonist midodrine.

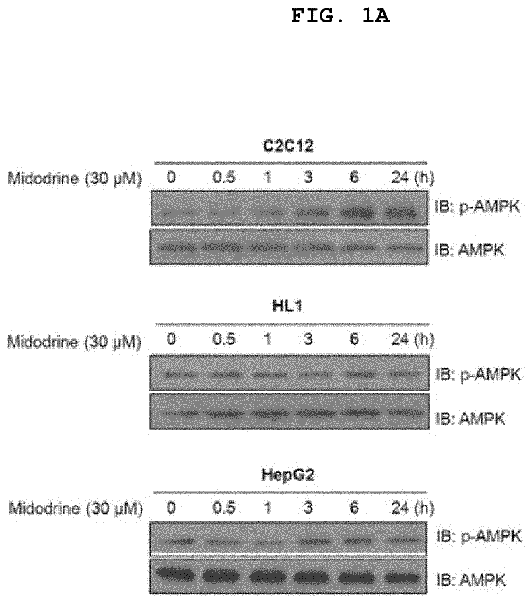

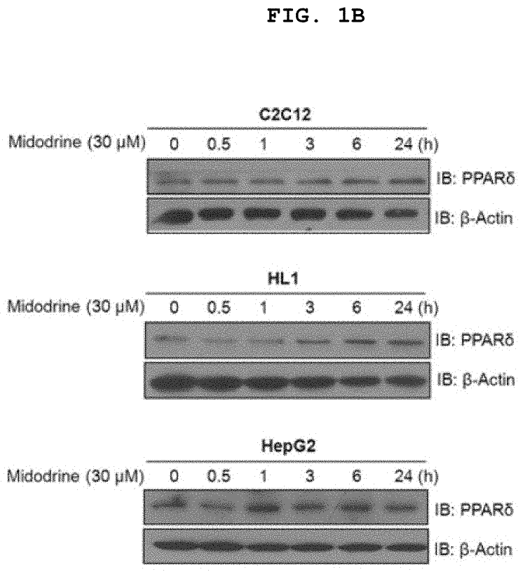

As seen from FIGS. 1A and 1B, AMPK was expressed in the skeletal muscle cells in response to the α₁-AR stimulation and the expression of phosphorylated AMPK was increased, depending upon the concentration of midodrine, suggesting that the α₁-AR stimulation is associated with AMPK activation. In addition, the expression and phosphorylation of AMPK in response to the α₁-AR stimulation by midodrine administration were also observed for the cardiac muscle cells.

Accordingly, because midodrine induces the change (AMPK activation) in cardiac muscle that also observed on skeletal muscle in response to midodrine—in addition to the previously known improvement of cardiac muscle contractility, it can be seen that the α₁-AR stimulation exhibits an additional exercise mimetic effect in the cardiac muscle. That is to say, although only the increase in the contractile force of cardiac muscle was known previously through in-vitro experiments, an exercise mimetic effect due to AMPK activation and phosphorylation in skeletal and cardiac muscle cells was demonstrated through this example.

2-2. Effect of α₁-AR Agonist Administration on Cardiac Function, Body Weight and Blood Pressure in Animal Model In Vivo

If midodrine administration leads to increase in AMPK, PPAR-δ and PGC-1α in skeletal muscle in vivo, it may be thought that the cascade of AMPK-PPAR-δ-PGC-1α activation with proven exercise mimetic effect in the skeletal muscle will also improve the contractility of cardiac muscle. Therefore, cardiac function and body weight were investigated in vivo.

Cardiac Function and Body Weight

As seen from Table 1 below, the echocardiographic data for 8-week-old rats revealed that the left ventricular performance of the midodrine-administered rat (group II) and the atenolol-administered rat (group III) was higher than that of the unadministered control (group IV).

The left ventricular mass was the lowest for the group II but no significant difference was observed in cardiac mass between the groups. The group III showed the highest body weight.

TABLE 1

| | Midodrine | Atenolol | Control | |
|---|---|---|---|---|
| Parameters | (group II) | (group III) | (group IV) | p-Value |
| Diastolic left ventricular | 1.46 ± 0.13^{a} | 1.50 ± 0.13^{a} | 1.45 ± 0.11^{a} | 0.3256 |
| diaphragm, mm | | | | |
| Diastolic left ventricular wall, mm | 1.54 ± 0.13^{a} | 1.67 ± 0.16 | 1.54 ± 0.15^{a} | 0.0046 |
| Diastolic left ventricular inner | 6.06 ± 0.42^{a} | 6.33 ± 0.35^{a} | 6.37 ± 0.72^{a} | 0.1082 |
| size, mm | | | | |
| Systolic left ventricular inner size, | 3.32 ± 0.54^{a} | 3.43 ± 0.28^{a} | 3.82 ± 1.01 | 0.0445 |
| mm | | | | |
| Left ventricular fractional | 45.48 ± 6.25^{a} | 45.70 ± 5.82^{a} | 38.77 ± 8.59 | 0.0019 |
| shortening, % | | | | |
| Left ventricular ejection | 81.55 ± 6.12^{a} | 82.00 ± 5.31^{a} | 73.87 ± 10.13 | 0.0007 |
| fraction, % | | | | |
| Left ventricular mass according to | 1.04 ± 0.06 | 1.10 ± 0.07^{a} | 1.07 ± 0.12^{a} | 0.0313 |
| ASE, g | | | | |
| Body weight, g | 238.24 ± 11.69^{a} | 296.46 ± 16.73^{b} | 236.20 ± 7.58^{a} | 0.009 |
| Cardiac weight, g | 1.47 ± 0.16^{a} | 1.47 ± 0.15^{a} | 1.78 ± 0.35^{a} | 0.062 |

2-3. Expression of AMPK, PPAR-δ and PGC-1α Proteins in Cardiac Muscle, Skeletal Muscle, Aorta and Liver

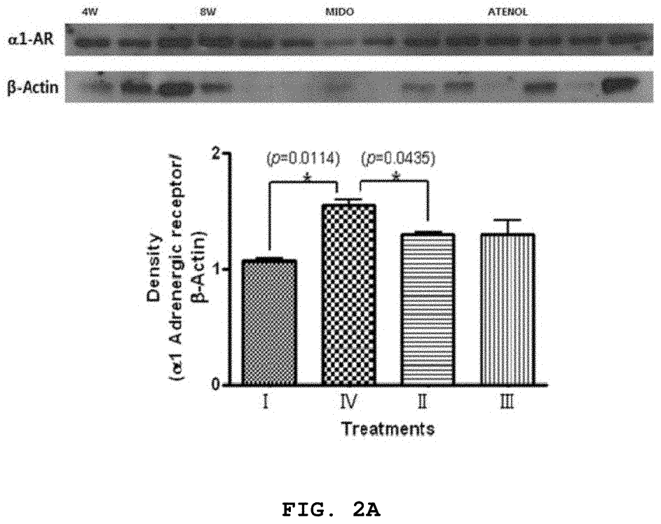

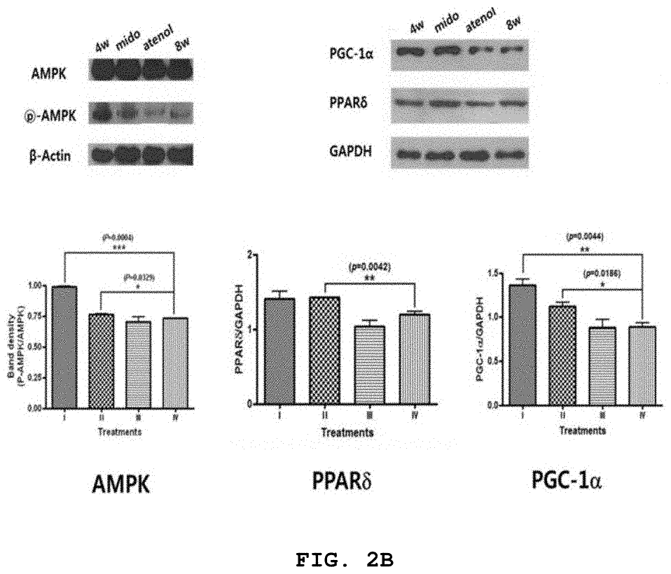

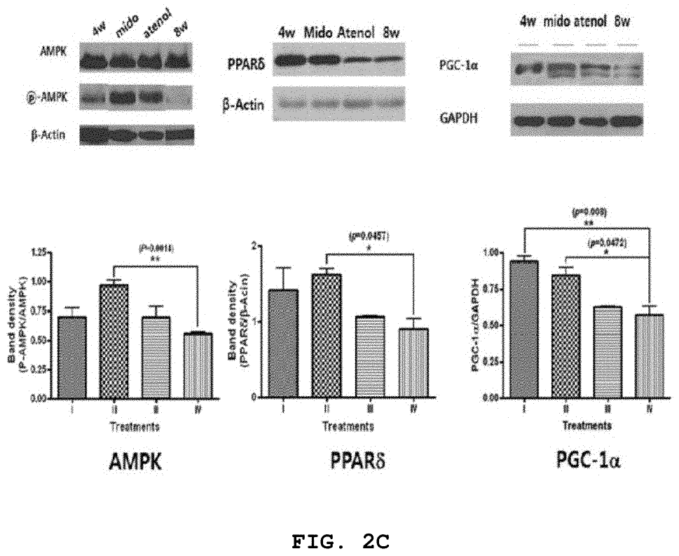

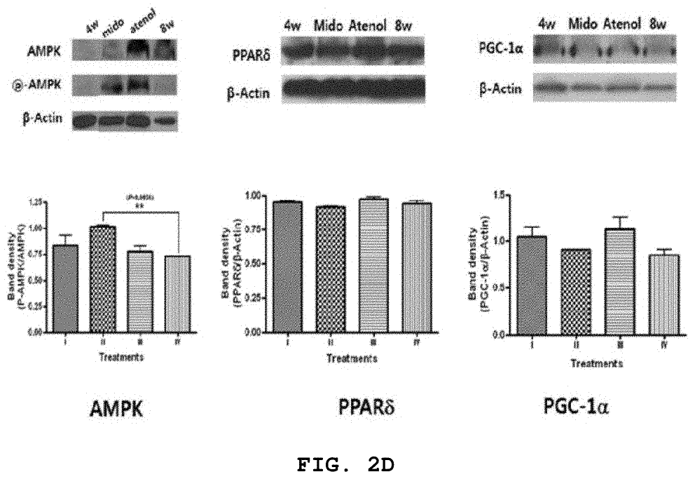

In order to confirm the presence of α₁-AR in skeletal muscle, the expression of the α₁-AR protein in skeletal muscle of the 4-week-old basal control group rat (I), the midodrine-administered rat (II), the atenolol-administered rat (III) and the 8-week-old unadministered control group rat (IV) was investigated by western blotting. As shown in FIG. 2A, the group IV showed the highest α₁-AR expression.

In addition, the expression of the AMPK, PPAR-δ and PGC-1α proteins in the cardiac muscle, skeletal muscle and liver was investigated by western blotting. As shown in FIG. 2B (cardiac muscle), FIG. 2C (skeletal muscle) and FIG. 2D (liver), in the cardiac muscle, the expression of the phosphorylated AMPK protein was higher for the group I than the group IV (p<0.05). The midodrine-treated group II showed significantly higher AMPK expression in the cardiac muscle and skeletal muscle than the control group IV, whereas the atenolol-treated group III did not show higher AMPK protein expression in the organs as compared to the control group IV of the same age.

It is to be noted that the increase of PGC-1α and PPAR-δ is much larger than the increase of AMPK in the cardiac muscle, whereas the increase of PGC-1α and PPAR-δ is much less than the increase of AMPK in the skeletal muscle. This means that, whereas the exercise mimetic effect induces AMPK activation and PGC-1α and PPAR-δ expression in the cardiac muscle, PGC-1α and PPAR-δ are not increased as much as compared to AMPK in the skeletal muscle.

2-4. Expression of Angiotensin II AT Receptor, eNOS, SOD, NADPH, TNFα and MCP Genes in Aorta and Heart

The mRNA expression level of the genes related with vasoconstriction, nitric oxide production, oxidative stress and inflammation in the aorta and cardiac muscle tissue was investigated by northern blot for the 4-week-old basal rat (I), midodrine-administered rat (II), atenolol-administered rat (III) and 8-week-old unadministered control group rat (IV).

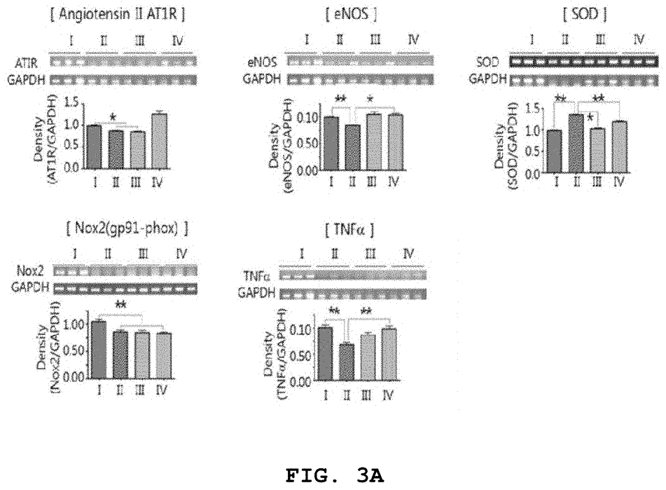

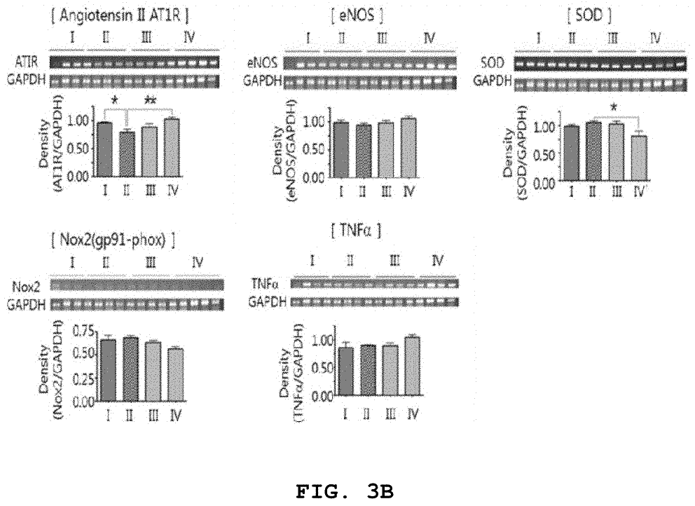

As seen from FIG. 3A, the expression of angiotensin II AT1R mRNA in the aorta was lower for the groups II and III as compared to the two control groups (groups I and IV). The expression of eNOS was the lowest in the group II but no significant difference from other groups was observed. The SOD expression was the highest in the group II. The expression of the NAD(P)H oxidase gp91-phox (Nox-2) mRNA was higher for the group I but there was no difference between the 8-week-old spontaneously hypertensive rats regardless of drug administration. The TNFα expression was the lowest in the group II, moderate in the group III, higher in the group IV and the highest in the group I.

Also, as seen from FIG. 3B, the expression of angiotensin II AT1R mRNA in the cardiac muscle was the lowest in the group II, similarly to the result for the aorta, and moderate in the group III. There was no difference in the eNOS expression among the groups. SOD was expressed at a significantly higher level in the group II as compared to the group IV. In contrast to the aortic (vascular) tissue, there was no difference in the expression of the NAD(P)H oxidase gp91-phox (Nox-2) mRNA between the groups regardless of drug administration. The expression of TNFα in the cardiac muscle was not decreased in response to the midodrine administration.

2-5. Effect of α₁-AR Agonist Administration on Cytokines, Reactive Oxygen Species, Adiponectin and Fat Profile

The effect of long-term administration of midodrine on the expression of cytokines (IL and TNFα), reactive oxygen species (ROS) and adiponectin and the fat profile was investigated. The result is given in Table 2.

TABLE 2

| | Basal control | Midodrine | Atenolol | Control | |
|---|---|---|---|---|---|
| | (Group I) | (Group II) | (Group III) | (Group IV) | p-value |
| IL-1β (μg/ml) | 0.22 ± 0.20^{a} | 0.46 ± 0.422^{a} | 2.02 ± 1.53^{a} | 1.23 ± 1.45^{a} | 0.194 |
| IL-1α (μg/ml) | 1.94 ± 1.09^{a} | 0.14 ± 0.32^{b} | 5.51 ± 2.64^{c} | 0.15 ± 0.35^{b} | 0.004 |
| IL-6 (μg/ml) | 4 50 ± 0.20^{a} | 4.82 ± 0.49^{a} | 5.62 ± 1.42^{a} | 5.48 ± 1.15^{a} | 0.360 |
| TNF-α(μg/ml) | 3.60 ± 0.65^{a} | 2.64 ± 1.64^{a} | 8.35 ± 1.36^{b} | 5.36 ± 1.94^{a} | 0.006 |
| IL-4 (μg/ml) | 1.72 ± 0.26^{a} | 1.84 ± 0.21^{a} | 2.94 ± 0.90^{b} | 2.28 ± 0.50^{ab} | 0.030 |
| IL-10 (μg/ml) | 12.34 ± 0.89^{a} | 12.15 ± 0.37^{a} | 15.33 ± 2.79^{b} | 13.52 ± 1.66^{b} | 0.094 |
| ROS (ng/ml) | 186.8 ± 25.7^{a} | 204.9 ± 27.5^{a} | 223.1 ± 45.4^{a} | 254.8 ± 16.8^{a} | 0.254 |
| Adiponectin | 74796.6 ± 13898.0^{a} | 7585.8 ± 182.3^{b} | 6136.5 ± 574.7^{bc} | 5455.8 ± 709.7^{c} | <0.001 |
| (ng/ml/g) | | | | | |

As seen from Table 2, the level of the proinflammatory cytokines (IL-1β and IL-6) was not significantly different between the groups. The level of IL-1α was lower in the group II than in the group I but was significantly increased in the group III. The TNF-α expression was significantly increased in the group III as compared to other groups. The expression of IL-4 and IL-10 was slightly higher in group III as compared to other groups.

The level of reactive oxygen species (ROS) was lower in the midodrine-administered group II and the atenolol-administered group III than the 8-week-old unadministered control group IV, although the difference was not statistically significant.

In order to investigate how the α₁-AR stimulation activates AMPK, the level of adiponectin was measured. The group II showed a significantly higher serum adiponectin level per the weight of visceral fat as compared to the group IV.

The blood lipid profile was as shown in Table 3. Although total cholesterol, LDL cholesterol and HDL cholesterol were higher in the control groups (groups I and IV) as compared to the drug-administered groups (groups II and IV), there was no significant difference in the triglyceride level between the groups.

TABLE 3

| Lipids, | Basal control | Midodrine | Atenolol | Control | |
|---|---|---|---|---|---|
| (SI/L) | (Group 1) | (Group II) | (Group III) | (Group IV) | p-value |
| Total cholesterol | 1.69 ± 0.29 | 0.73 ± 0.15^{a} | 1.01 ± 0.27^{a} | 1.66 ± 0.17 | <0.001 |
| LDL cholesterol | 0.43 ± 0.06 | 0.15 ± 0.04^{a} | 0.15 ± 0.03^{a} | 0.40 ± 0.03 | <0.001 |
| HDL cholesterol | 0.81 ± 0.08 | 0.49 ± 0.11^{a} | 0.52 ± 0.04^{a} | 0.87 ± 0.15 | <0.001 |
| Triglyceride | 0.83 ± 0.33^{a} | 0.43 ± 0.12^{a} | 0.77 ± 0.33^{a} | 0.77 ± 0.31^{a} | 0.092 |

2-6. Change in Expression of Mitochondrial Oxidases

The enzyme activity of succinate dehydrogenase (SDH) which is involved in the mitochondrial oxidation process (TCA cycle) was measured in skeletal muscle. As seen from FIG. 4A, the midodrine-administered group II showed the highest activity.

Also, cytochrome c oxidase which is an enzyme involved in the mitochondrial electron transport chain in the skeletal muscle tissue was immunohistochemically stained. As seen from FIG. 4B, the enzyme was the most increased in the midodrine-administered group II.

From these results, it can be seen that midodrine provides a superior effect in metabolism.

2-7. Comparison of ATP Level in Tissues

The level of ATP in the heart, skeletal muscle and liver was measured by ELISA for the 4-week-old basal rat (I), midodrine-administered rat (II), atenolol-administered rat (III) and 8-week-old unadministered control group rat (IV).

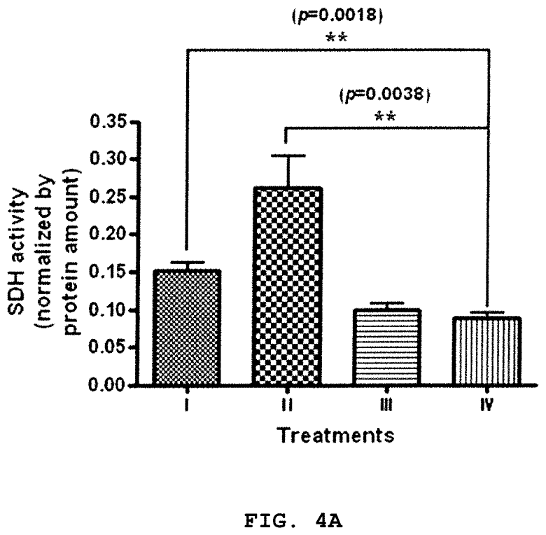

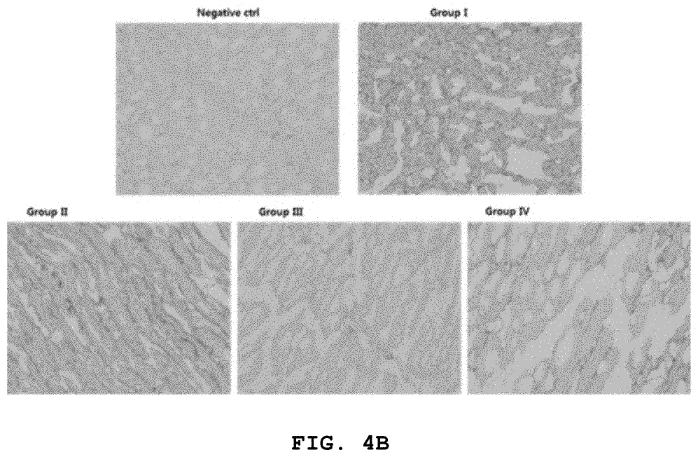

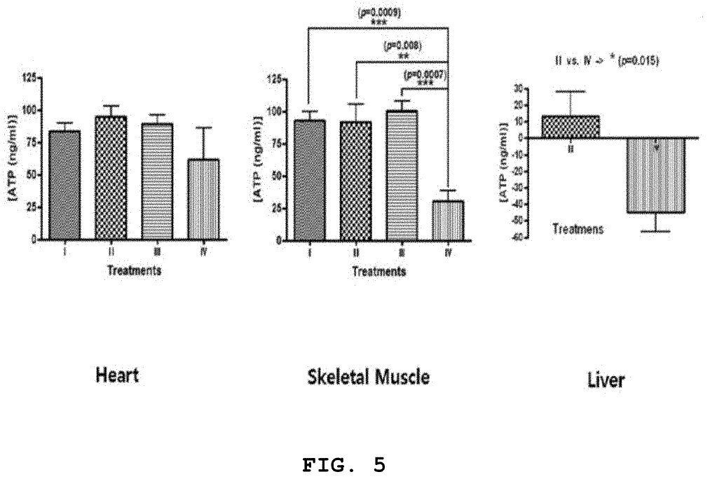

As seen from FIG. 5, the heart tissue of the group treated with midodrine or atenolol showed a higher ATP level than the control group SHRs despite the higher cardiac contraction activity.

2-8. Diabetes-Treating Effect

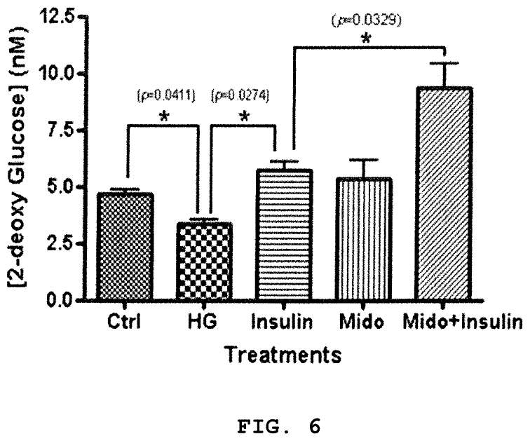

The effect of midodrine on glucose uptake by insulin in mouse skeletal muscle cells (C2C12 cells) was investigated. As seen from FIG. 6, it was confirmed that the glucose uptake was significantly increased when insulin and midodrine were treated in combination as compared to when insulin was treated alone. Accordingly, it can be seen that midodrine is effective in treating diabetes.

2-9. Obesity-Treating Effect

Inhibition of Adipocyte Differentiation

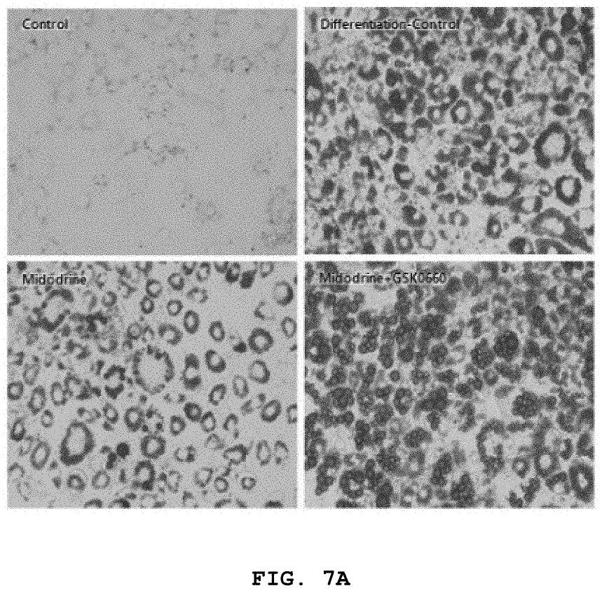

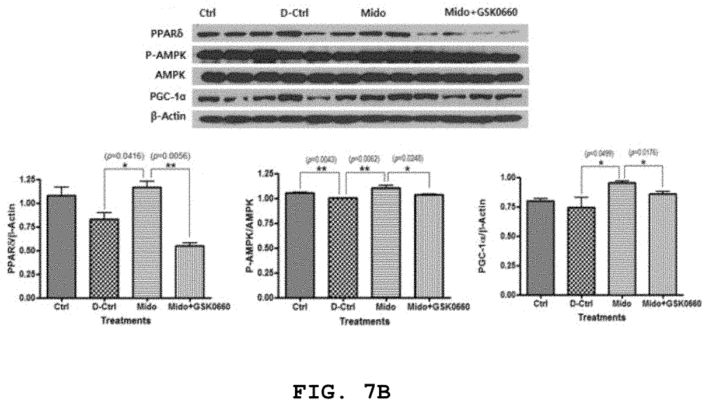

When preadipocytes (3T3-L1) were treated with midodrine, differentiation into adipocytes was inhibited as shown in FIG. 7A (left bottom). Also, the expression of the PPARδ, p-AMPK and PGC-1α proteins, which inhibit fat synthesis and accumulation, was increased as shown in FIG. 7B.

Reduction of Body Weight and Abdominal Fat

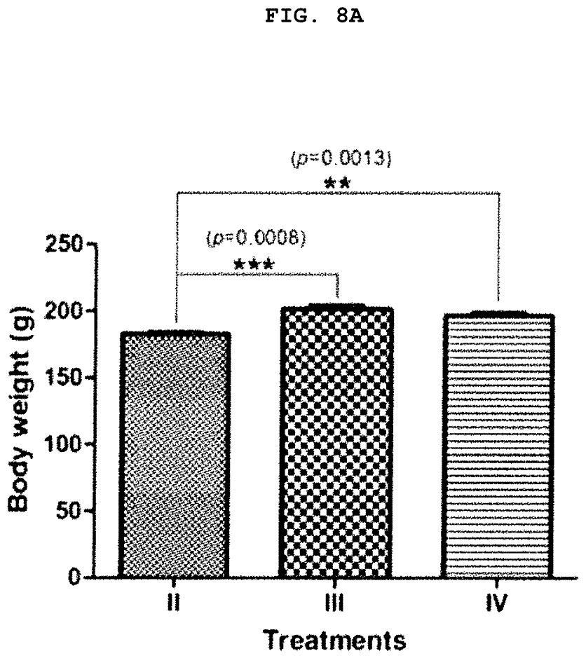

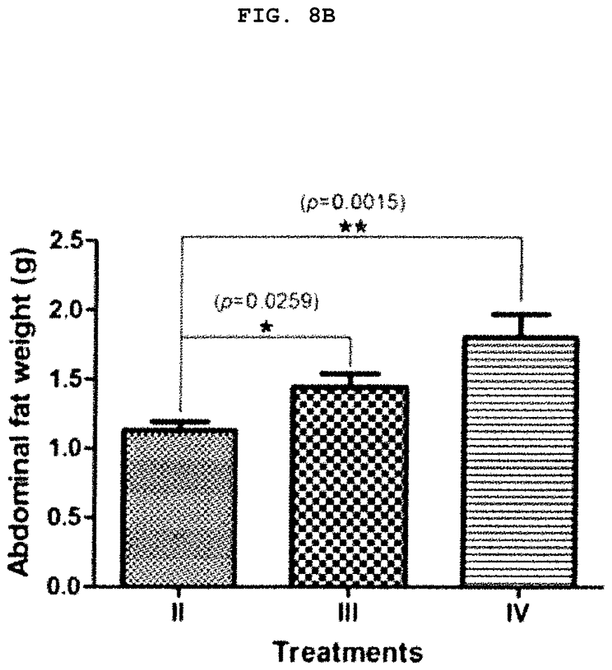

Body weight and abdominal fat were measured and compared for the midodrine-administered rat (II), atenolol-administered rat (III) and 8-week-old unadministered hypertension control group rat (IV). As seen from FIG. 8, the midodrine administration resulted in reduction of body weight and abdominal fat.

Accordingly, it can be seen from these results that midodrine is effective in treating obesity.

2-10. Inflammation-Treating Effect

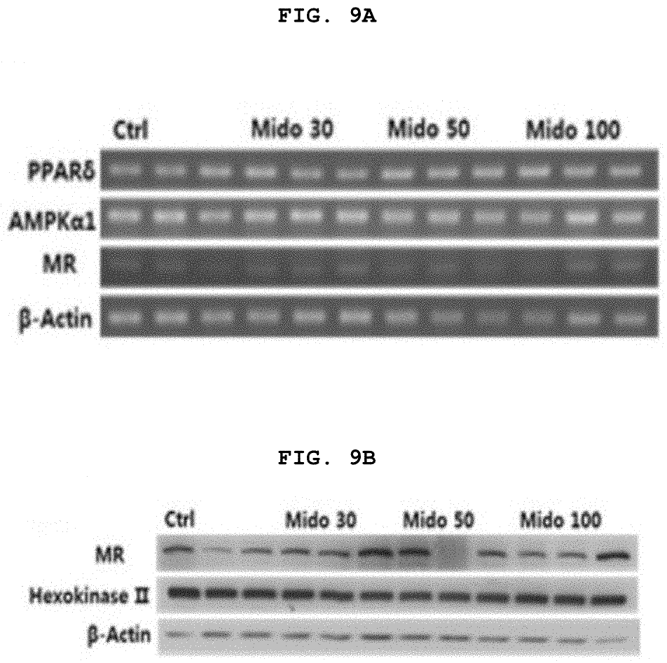

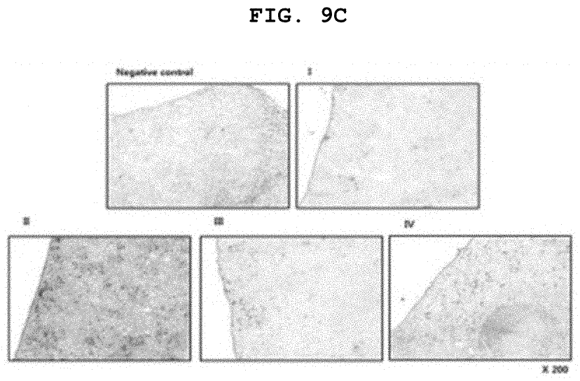

When the Raw 264.7 macrophages were treated with midodrine, as seen from FIG. 9, the expression of PPARδ, AMPKα₁ and mannose receptor (MR) mRNAs was increased (A) and the expression of the hexokinase II protein was decreased (B). Also, the increased expression of mannose receptor in macrophages localized in the subcapsular portion of the spleen was confirmed.

Because the expression of the mannose receptor (MR), which is the M2 phenotype receptor exhibiting anti-inflammatory effect in inflammation-related macrophages, was increased whereas the expression of hexokinase II, which is increased in M1 phenotype macrophages, was decreased, it can be seen that midodrine is effective in treating inflammations.

### Example 3: Effect of Various α₁-Adrenergic Receptor Agonists

Compounds similar to midodrine were newly synthesized as α₁-AR agonists and their effect of inducing AMPK activation and PPAR-δ and PGC-1α expression in skeletal muscle cells by stimulating α₁-AR was confirmed. It was verified that the various α₁-AR agonists can induce an exercise mimetic effect.
Compound 5: 2-amino-N-(2-(2,5-dimethoxyphenyl)-2-hydroxyethyl)-3-phenylpropanamide
Compound 7: 2-amino-N-(2-(2,5-dimethoxyphenyl)-2-hydroxyethyl)propanamide
Compound 8: 2-amino-N-(2-(3,5-dimethoxyphenyl)-2-hydroxyethyl)acetamide hydrochloride
Compound 9: 2-amino-N-(2-(5-ethyl-2-methoxyphenyl)-2-hydroxyethyl)acetamide hydrochloride
Compound 10: 2-amino-N-(2-hydroxy-2-phenylethyl)acetamide hydrochloride

Specifically, after treating differentiating C2C12 cells with the compounds at different concentrations (10 μM, 30 μM, 50 μM), the expression of activated (phosphorylated) AMPK (p-AMPK), PPAR-δ and PGC-1α was investigated by western blot.

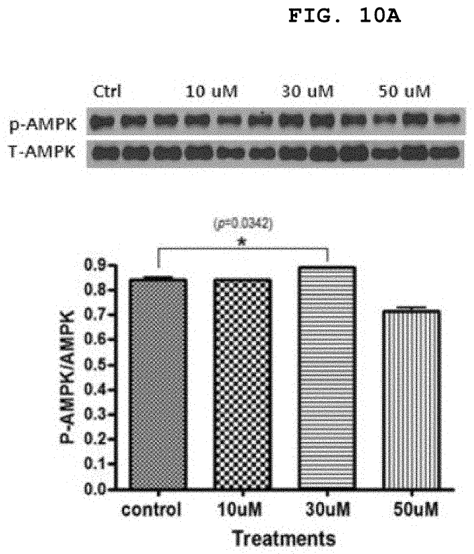

As seen from FIGS. 10A and 10B, Compound 5 showed significantly increased expression of activated (phosphorylated) AMPK (p-AMPK) and PPAR-δ at the intermediate concentration of 30 μM as compared to the control group (untreated group).

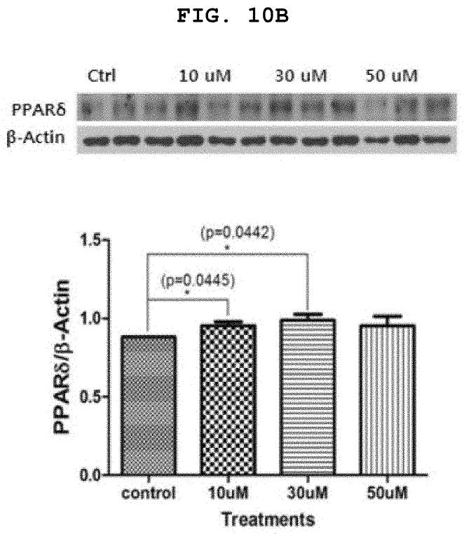

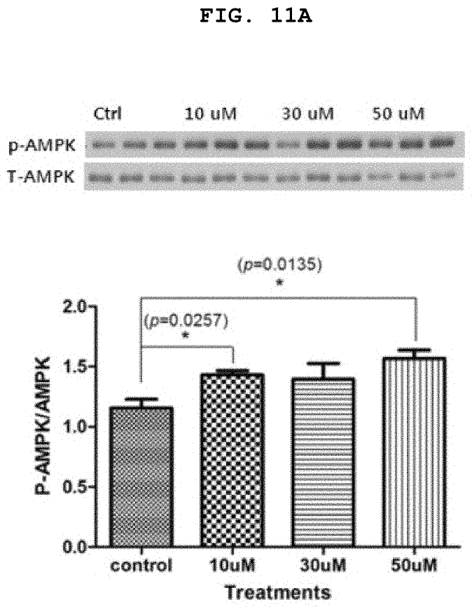

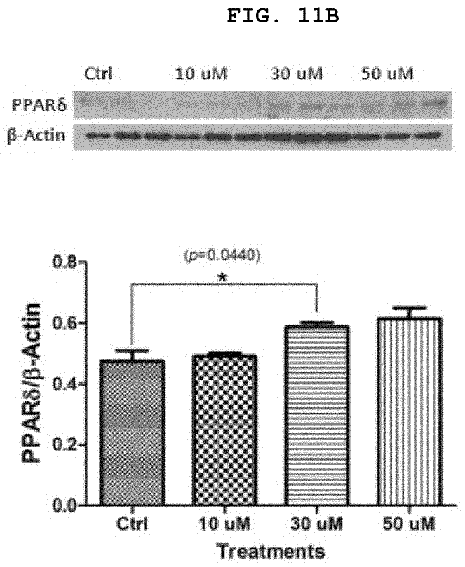

As seen from FIGS. 11A and 11B, Compound 7 showed concentration-dependent increase in the expression of p-AMPK and PPAR-δ proteins. In particular, for p-AMPK, the 10 μM treatment group and the 50 μM treatment group showed significant increase as compared to the control group. For PPAR-δ, the 30 μM treatment group showed significant increase as compared to the control group.

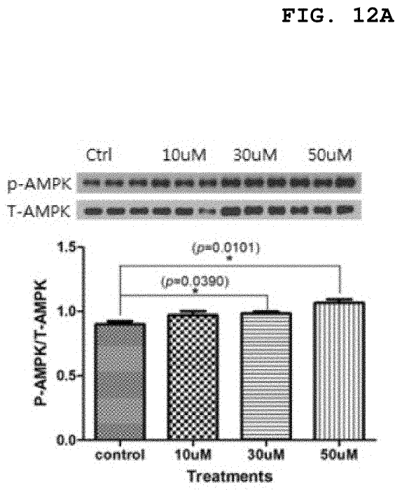

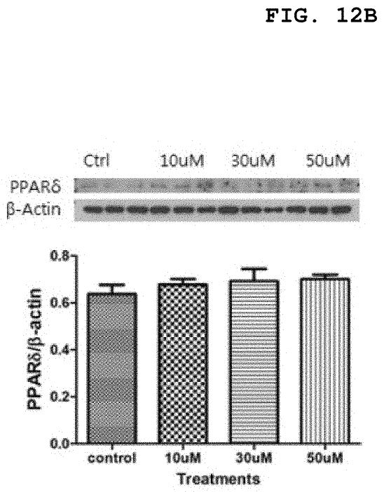

As seen from FIGS. 12A-12C, Compound 8 showed overall increase of p-AMPK, PPAR-δ and PGC-1α. In particular, the expression of p-AMPK and PGC-1α was significantly increased at the concentration of 50 μM.

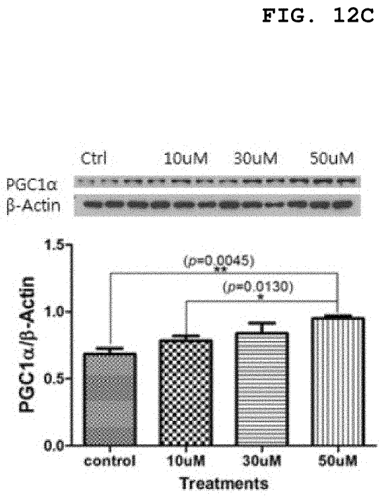

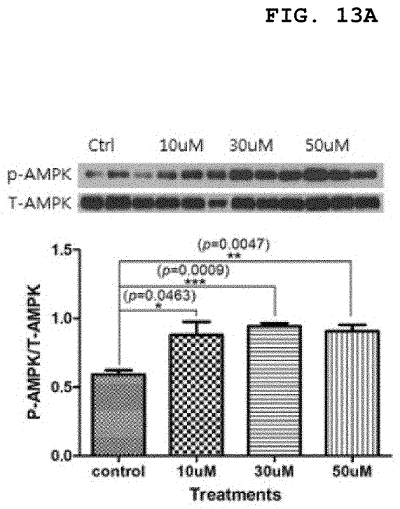

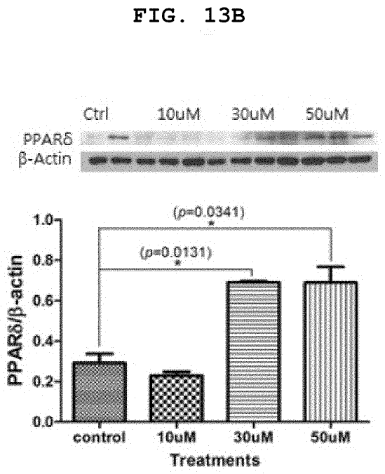

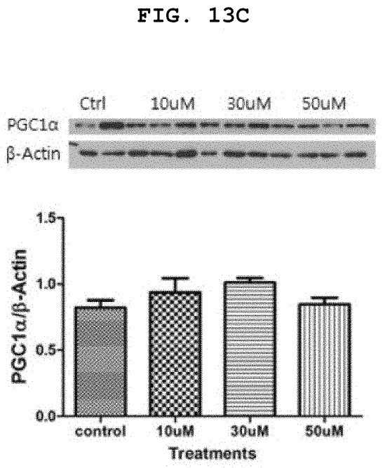

As seen from FIGS. 13A-13C, Compound 9 showed overall increase of p-AMPK, PPAR-δ and PGC-1α. In particular, the expression of p-AMPK and PGC-1α was significantly increased at the concentrations of 30 μM and 50 μM.

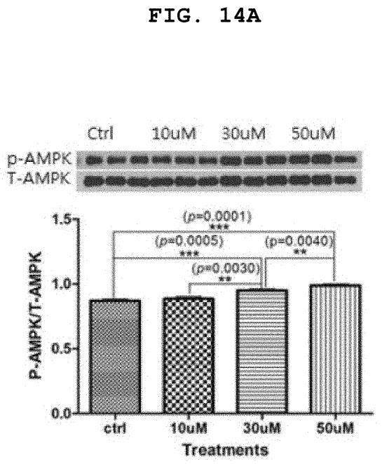

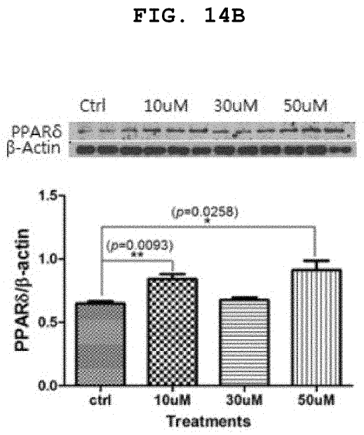

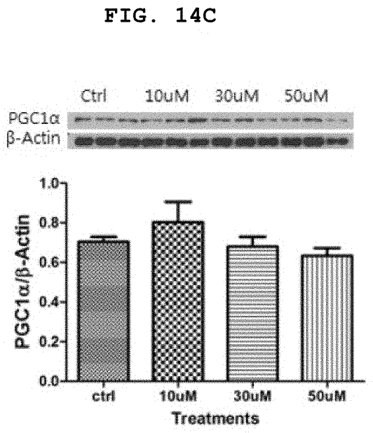

As seen from FIGS. 14A-14C, Compound 10 showed significantly increased p-AMPK expression at 30 μM and 50 μM and significantly increased PPARδ expression at 10 μM and 50 μM.

According to the above results, it can be seen that α₁-AR stimulation by the α₁-AR agonist induces an exercise mimetic effect in skeletal muscle, liver and blood vessels even without exercise, improves left ventricular ejection fraction without cardiac hypertrophy or blood pressure increase, reduces inflammation and changes biochemical responses during the initial hypertensive development of spontaneously hypertensive rat. This is directly related with stimulated cardiac muscle contraction, cardiac motion-like effect and AMPK activation.

Those skilled in the art will appreciate that the conceptions and specific embodiments disclosed in the foregoing description may be readily utilized as a basis for modifying or designing other embodiments for carrying out the same purposes of the present disclosure. Those skilled in the art will also appreciate that such equivalent embodiments do not depart from the spirit and scope of the disclosure as set forth in the appended claims.

1. A method for inducing an exercise-like effect, comprising administering an α1-adrenergic receptor agonist or a pharmaceutically acceptable salt thereof to a subject.

2. The method according to claim 1, wherein the α1-adrenergic receptor agonist induces an activation of AMPK.

3. The method according to claim 1, wherein the α1-adrenergic receptor agonist induces an expression of PPAR-δ or PGC-1α.

4. The method according to claim 1, wherein the α1-adrenergic receptor agonist is (R/S)—N-[2-(2,5-dimethoxyphenyl)-2-hydroxyethyl]glycinamide.

5. The method according to claim 1, wherein the exercise-like effect is an effect of treating a disease requiring an activation of AMPK.

6. The method according to claim 1, wherein the α1-adrenergic receptor agonist induces the exercise-like effect in organs comprising skeletal muscle, cardiac muscle, liver, and pancreas.

7. The method according to claim 1, wherein the α1-adrenergic receptor agonist induces the exercise-like effect in a patient suffering from diseases selected from a group consisting of cardiovascular diseases, metabolic diseases, and inflammatory diseases.

8. The method according to claim 7, wherein the metabolic diseases are selected from a group consisting of hypertension, hyperlipidemia, diabetes, obesity, arteriosclerosis and fatty liver.

9. The method according to claim 2, wherein the activation of AMPK comprises an expression of phosphorylated AMPK.

10. The method according to claim 1, wherein the α1-adrenergic receptor agonist is selected from a group consisting of 2-amino-N-(2-(2,5-dimethoxyphenyl)-2-hydroxyethyl)-3-phenylpropanamide, 2-amino-N-(2-(2,5-dimethoxyphenyl)-2-hydroxyethyl)propanamide, 2-amino-N-(2-(3,5-dimethoxyphenyl)-2-hydroxyethyl)acetamide hydrochloride, 2-amino-N-(2-(5-ethyl-2-methoxyphenyl)-2-hydroxyethyl)acetamide hydrochloride, and 2-amino-N-(2-hydroxy-2-phenylethyl)acetamide hydrochloride.

11. The method according to claim 1, wherein the exercise-like effect comprises an effect selected from a group consisting of: a physiological effect exerted during exercise comprising improvement in cardiac function or increase in contractile force; increase in insulin sensitivity and oxidative phosphorylation of muscles; decrease in cholesterol; decrease in fat accumulation and body weight; and decrease in blood inflammation.
